(19) United States
(12) Patent Application Publication
(10) Pub. No.: US 2020/0072820 A1
(43) Pub. Date: Mar. 5, 2020

(54) Method of Selecting for Antibodies

(71) Applicant: Oxford Genetics Limited, Oxford Oxfordshire (GB)

(72) Inventors: Ryan Cawood, Oxford Oxfordshire (GB); Thomas Payne, Oxford Oxfordshire (GB); Richard Parker-Manuel, Oxford Oxfordshire (GB)

(21) Appl. No.: 16/492,777

(22) PCT Filed: Mar. 14, 2018
(86) PCT No.: PCT/GB2018/050645

§ 371 Date: Sep. 10, 2019

(30) Foreign Application Priority Data
Mar. 15, 2017 (GB) 1704115.3

(51) Int. Cl. G01N 33/537; C07K 16/42; G01N 33/566; G01N 33/531; G01N 33/68; C40B 30/04; C40B 40/02; C40B 40/10; C12N 15/10; G01N 33/50

(52) U.S. Cl. CPC G01N 33/537; C07K 16/42; G01N 33/566; G01N 33/531; G01N 33/6854; C40B 30/04; G01N 2500/04; C40B 40/10; C12N 15/1037; G01N 33/6845; G01N 33/5032; G01N 2500/10; C40B 40/02

(57) ABSTRACT

The present invention relates to a method for identifying specific binding partners (e.g. antibodies or antibody mimetics) which bind to a desired target polypeptide. In particular, the method involves expressing a library of antibodies or antibody mimetics in a population of mammalian cells, wherein each cell in the population of cells displays the target polypeptide on the outer surface of the cell, and identifying or isolating cells within the population of cells to which antibodies or antibody mimetics are bound.

### CROSS-REFERENCE

This application is a section 371 U.S. National phase of PCT/GB2018/050645, filed Mar. 14, 2018 which claims priority from GB 1704115.3, filed Mar. 15, 2017, both which are incorporated by reference in its entirety.

### FIELD OF THE INVENTION

The present invention relates to a method for identifying specific binding partners (e.g. antibodies or antibody mimetics) which bind to a desired target polypeptide. In particular, the method involves expressing a library of antibodies or antibody mimetics in a population of mammalian cells, wherein each cell in the population of cells displays the target polypeptide on the outer surface of the cell, and identifying or isolating cells within the population of cells to which antibodies or antibody mimetics are bound.

### BACKGROUND OF THE INVENTION

Since the invention of hybridoma technology in 1986, monoclonal antibodies have emerged as powerful and versatile biological therapeutics, combining target selectivity, potency, good biological and delivery half-lives and relatively simple large scale manufacture. Today, almost fifty monoclonal antibodies are licensed for medical use in the US and Europe, and many others are in development. They are used in the treatment of a broad range of diseases, including inflammation (e.g. rheumatoid arthritis, Crohn's disease, ulcerative colitis, etc.), organ transplantation, asthma, cancer and leukaemia, viral and bacterial infections, aberrant blood clotting and many others.

In medical terms, monoclonal antibodies are usually well-tolerated, with few side-effects, and can have life-changing medical benefits. However, the increasing appreciation of their therapeutic potential has created a rising demand for new monoclonal antibodies against a broad range of targets. This in turn has highlighted the difficulties faced in defining monoclonal antibodies with sufficient potency against challenging targets, most notably against molecules on the cell surface such as integrated membrane proteins. These targets need to retain their physiological configuration during antibody selection, and this severely restricts the strategies that can be used to produce monoclonal antibodies that recognise them (Jones, M. et al., Scientific Reports, 6, 26240 (2016)). It is particularly challenging to define naturally-occurring human antibodies that bind to human targets, because of clonal deletion of many self-recognising antibodies during development.

GPCRs have been historically hard to produce monoclonal antibodies against, due to their need to stay membrane-associated in order to retain their configuration. GPCRs constitute the largest family of membrane protein in humans and are responsible for cellular responses to hormones and neurotransmitters, light sensing, olfaction and taste. About half of current low molecular weight drugs target GPCRs; however, few monoclonal antibodies are in development—even for research—because they are elusive to target. One good example is DRD1 (D1 subtype of the dopamine receptor) which regulates neuronal growth and development, some behavioural responses and modulates DRD2 activity. DRD1 deregulation is thought to play a role in schizophrenia, Huntington's Disease, Parkinson's Disease, hypertension, Alzheimer's Disease and many others. The GPCR market is currently estimated at $1.6bn (http://www.transparencymarketresearch.com/g-protein-coupled-receptors-market.htm I). Of the 47 drugs approved that target DRD1, none are monoclonal antibodies. This illustrates the challenge of making effective monoclonal antibodies recognising membrane antigens in their native configuration.

Cancer checkpoint inhibitor antibodies are currently the most exciting new aspect of cancer research, with several agents already licensed against a variety of targets. The market for these is predicted to reach a staggering $19bn in 2022 (http://immunecheckpoint-europe.com/partner/sponsorship-opportunities/). The one feature these targets share is that they are all membrane antigens (e.g. PD1, PDL1, CTLA4, etc.).

Antibody display may be used to screen for antibodies against a particular target polypeptide. Existing technologies for antibody display include phage display, yeast display, mammalian display, ribosome display, cis-activity based (CIS) display and covalent antibody display (CAD). These technologies all have the same limitation in that membrane target polypeptides (baits') are not presented in their native folded membrane-bound state.

The classical method for high-throughput screening for protein interactions is phage display. In this system, an antibody library is fused to the gene for a bacteriophage coat protein. The library is then transformed into an E. coli host strain (using a phagemid), resulting in a population of phage particles, each containing the sequence for an antibody within its genome and displaying the protein itself on its surface. At first, the phage library is screened with the targeted antigen, immobilised to a surface. Then, unbound phages are washed away; bound phages are recovered and infected into bacteria, and subsequently amplified for library enrichment. This process is normally repeated several times, yielding sequences of gradually improving affinity for the target. The protein sequence (and level of emerging consensus or homology) of phage in the library can be determined by isolating individual colonies and sequencing their DNA in the appropriate region.

Other systems use a similar concept: for instance, Isogenica's proprietary CIS in vitro display-technology uses the ability of a protein called RepA to bind to its own DNA sequence, allowing it to act as a linker between phenotype and genotype. The advantage of this system is that it facilitates rapid recovery of the antibody encoding DNA sequence after the selection step. However, a significant disadvantage of this system is that the bait protein must be immobilised to a solid support during the in vitro selection step. This precludes the targeting of certain proteins such as large multi-pass membrane proteins.

Cell-surface display is the expression of antibody proteins on the surface of living cells by fusing them to functional components of cells which are exposed to the extracellular milieu. The principle of cell surface display is analogous to phage display, with the recombinant antibody anchored to the cell's surface and the encoding DNA residing within the cell. One advantage of cell-surface display is that cells are large enough to be screened by flow cytometry. In contrast to acellular approaches, the antigen, labelled with a fluorophore, is incubated with the cell-displayed antibody library in solution and then any antigen binding cells are then isolated by fluorescence-activated cell sorting (FACS). Display strategies have been developed for use with bacterial, yeast and mammalian cells. The advantage of using mammalian cells is their ability to express and fold the antibodies in one's library with high fidelity and even with proper post-translational modifications.

One disadvantage of cellular display technologies is that only a relatively small library size is possible compared to acellular technologies due to the limitations of transfecting the library into cells.

The key disadvantage of mammalian cell display, however, is that the antigen has to be in solution. This limits the antigen to relatively small, hydrophilic proteins, essentially excluding large multi-pass membrane proteins, an important class of targets for antibody discovery. Attempts to address this include presenting the antigen in the context of membranous vesicles, but this approach is laborious and so far not very successful.

Chen Zhou's group have developed a mammalian display system for screening full-length antibody cDNA-based libraries (Zhou et al., Acta Biochimica et Biophysica Sinica, 42(8), 575-84.2010; US 2012/0101000). Their system expresses human antibody heavy and light chains together on the surface of mammalian cells; a transmembrane domain from platelet-derived growth factor receptor is fused to the heavy chain to anchor the antibody to the membrane of the cell that expressed it. A human heavy chain (IgG-1) library was constructed separately by RT-PCR amplifying the variable domain from PBMCs and cloning it into a plasmid vector. A human light chain (kappa) library was similarly constructed and the system was used successfully to select antibodies against soluble target antigens. This demonstrates that it is possible to use full-length antibody libraries at the scale necessary to do screening against targets in mammalian cells. However, their approach cannot obtain antibodies against complex membrane bound targets (most commonly required) as it requires a soluble protein for bioselection.

### SUMMARY OF THE INVENTION

The invention aims to overcome one or more of the above-mentioned problems by providing a new and rapid strategy for bioselection of monoclonal antibodies which recognise proteins on cells, particularly integrated membrane proteins. The approach improves over existing strategies by expressing the antigen on the surface of cells which secrete a library of polypeptide binding partners, e.g. antibodies or antibody mimetics, and then isolating cells that self-label. The process may be repeated in order to allow library evolution, followed by affinity maturation of lead candidate polypeptide binding partners.

One advantage of this solution is that the membrane-bound target polypeptide passes through the proper cellular folding and membrane-insertion pathways before presentation on the surface of the cells. The segment of the membrane-bound target polypeptide presented to the polypeptide binding partners (e.g. antibodies/mimetics) in the library is the same as that which would be available to be bound in an in vivo (cell culture or therapeutic) setting. The ability to select binding partners (e.g. antibodies/mimetics) that bind to membrane proteins is important because membrane proteins in general, including immune checkpoints and G-protein coupled receptors, are key therapeutic targets.

### DESCRIPTION OF THE DRAWINGS

FIG. 1: Labelling of cells expressing the EpCAM protein with an anti-EpCAM single chain antibody. The bar graph shows the median fluorescence intensity of each population of cells after staining with a fluorescently-labelled anti HA-antibody and subsequent flow cytometry analysis. HEK293 cells were co-transfected with EpCAM and an anti-EpCAM single chain antibody (top bar). Controls included cells transfected with either EpCAM or the antibody alone or with empty vector (bottom).

FIG. 2: Cells expressing EpCAM and GFP were mixed with cells expressing EpCAM and anti-EpCAM scFv. The X-axis indicates the degree of scFv labelling whereas the Y-axis indicates GFP fluorescence intensity. Cells expressing GFP but not scFv appear in the upper left (UL) quadrant; cells self-labelled with scFv but negative for GFP appear in the lower right (LR) quadrant; GFP positive cells that have become trans-labelled with soluble scFv from the scFv expressing-cells appear in the upper right (UR) quadrant. At higher ratios of EpCAM-GFP to EpCAM-scFv cells (25:1 and 125:1), a small sub-population of self-labelled cells is observed before any detectable antibody has had chance to transfer to other cells in the population.

FIG. 3: One example of the method of the invention, showing antibodies recognising integrated membrane proteins. A. Co-expression of bait protein on human cell surface (open dot) alongside secreted scFv library (average one scFv per cell). B. A cell that expresses a scFV that binds to the bait becomes self-labelled. C. Cells are stained for surface-bound scFv with a fluorescent secondary antibody (starred). D. Fluorescent cells are sorted into individual wells of a microtiter plate by FACS. Supernatant binding activity is characterised and lead candidates are sequenced prior to affinity maturation.

FIG. 4: One example of a target polypeptide (bait) expression construct.

FIG. 5: One example of a scFv expression construct. Expression is driven by the spleen focus-forming virus (SFFV) promoter. The expression construct encodes a C-terminal Human influenza hemagglutinin (HA)-tag.

### DETAILED DESCRIPTION OF THE INVENTION

In one embodiment, the invention provides a method of identifying a cell which produces a specific binding partner which binds to a target polypeptide, the method comprising the steps:
(a) expressing a library of binding partners in a population of mammalian cells, wherein each binding partner comprises a core framework and a plurality of variable regions, each plurality of variable regions endowing that binding partner with a specific binding affinity for a target, wherein each binding partner is secreted from the cell in which it is produced, and wherein the target polypeptide is displayed on the outer surface of each cell in the population of mammalian cells; and
(b) isolating or identifying cells within the population of mammalian cells to which specific binding partners are bound,
   wherein the cells to which specific binding partners are bound are ones which produce specific binding partners which bind to the target polypeptide. Preferably, the specific binding partner is an antibody or an antibody mimetic.

In a further embodiment, the invention provides a method of identifying a cell which produces an antibody or antibody mimetic which binds to a target polypeptide, the method comprising the steps:
(a) expressing a library of antibodies or antibody mimetics in a population of mammalian cells, wherein each antibody or antibody mimetic is secreted from the cell in which it is produced, and wherein the target polypeptide is displayed on the outer surface of each cell in the population of mammalian cells; and
(b) isolating or identifying cells within the population of mammalian cells to which antibodies or antibody mimetics are bound,
   wherein the cells to which antibodies or antibody mimetics are bound are ones which produce antibodies or antibody mimetics which bind to the target polypeptide. Preferably, the target polypeptide is expressed within each cell in the population of cells, preferably from an expression construct.

Preferably, the method additionally comprises the step:
(c) sequencing (part of all of) the polynucleotide sequences in the isolated cells which encode the antibodies or antibody mimetics which bind to the target polypeptide.

The invention also provides a method of obtaining the nucleotide sequence of a specific binding partner (e.g. an antibody or antibody mimetic) which binds to a target polypeptide, the method comprising the steps of a method of the invention of identifying a cell which produces a specific binding partner which binds to the target polypeptide, and additionally comprising the step of sequencing (all or part of) the nucleic acid in the cell which encodes that specific binding partner.

The invention also provides a method of obtaining the amino acid sequence of a specific binding partner (e.g. an antibody or antibody mimetic) which binds to a target polypeptide, the method comprising the steps of a method of the invention of identifying a cell which produces a specific binding partner which binds to the target polypeptide, purifying that specific binding partner, and obtaining the amino acid sequence of (all or part of) that purified specific binding partner.

In yet a further embodiment, the invention provides a process for producing a population of cells, the process comprising the steps: transforming a first population of mammalian cells with
(a) a plurality of first expression constructs, the plurality of first expression constructs encoding a library of secretable binding partners (e.g. antibodies or antibody mimetics); and
(b) a second expression construct which encodes a desired target polypeptide the target polypeptide comprising a transmembrane domain,
   so as to produce a second population of mammalian cells, wherein each cell in the second population of mammalian cells secretes or is capable of secreting one or more binding partners (e.g. antibodies or antibody mimetics), and wherein each cell in the second population of mammalian cells displays or is capable of displaying the target polypeptide on the outer surface of the mammalian cell.

Preferably, the plurality of first expression constructs (and/or the second expression construct) is delivered to the population of mammalian cell by a virus (preferably a retrovirus, more preferably a lentivirus) which is capable of infecting the mammalian cells.

The method of the invention will, in general, be carried out in vitro or ex vivo. Each cell (or substantially each cell) in the population of mammalian cells displays the target polypeptide on the outer surface of the cell. The target polypeptide is not secreted into the media surrounding the cell; the target polypeptide remains bound to the cell.

The target polypeptide preferably comprises one or more transmembrane domains in order to locate the target polypeptide in the outer cell membrane of the cell. In one embodiment, the target polypeptide is an integrated membrane protein. Preferably, it is integrated directly in the outer membrane of the cell.

In one embodiment, the target polypeptide is a fusion polypeptide which comprises an antigenic polypeptide linked to a transmembrane domain (e.g. a platelet derived growth factor receptor domain). The transmembrane domain anchors the antigenic polypeptide in the cell membrane and allows the antigenic domain to be displayed. The amino acid sequence of the antigenic polypeptide and the transmembrane domain may be linked by a short amino acid linker, e.g. 1-10 or 1-20 amino acids. The target polypeptide may be a glycosylated polypeptide or a non-glycosylated polypeptide.

In some embodiments, the target polypeptide is a single-pass membrane protein or a multiple-pass membrane protein. In some embodiments, the target polypeptide comprises 1, 2, 3, 4, 5, 6, or 7 transmembrane domains.

In some embodiments, the target polypeptide is a G-protein coupled receptor (GPCR) (e.g. DRD1). In some embodiments, the target polypeptide is an immunotherapy target, e.g. CD19, CD40 or CD38. In some embodiments, the target polypeptide is a protein which increases/decreases proliferation of cells, e.g. a growth factor receptor. In some embodiments, the target polypeptide is an ion channel polypeptide.

In some preferred embodiments, the target polypeptide is an immune checkpoint molecule. Preferably, the immune checkpoint molecule is a member of the tumour necrosis factor (TNF) receptor superfamily (e.g. CD27, CD40, OX40, GITR or CD137) or a member of the B7-CD28 superfamily (e.g. CD28, CTLA4 or ICOS). Preferably, the immune checkpoint molecule is PD1, PDL1, CTLA4, Lag1 or GITR.

In some embodiments, the target polypeptide is not avidin or streptavidin. In other embodiments, the target polypeptide is displayed on the outer surface of the cell in a target polypeptide/MHC1 complex. In such an embodiment, the target polypeptide and the MHC1 may be both over-expressed within the cells in order to achieve presentation of the target polypeptide in the MHC groove.

The target polypeptide is preferably expressed within each cell of the population of cells. The target polypeptide is preferably expressed from an expression construct. This expression construct may be integrated into the host cell genome or it may be present in a (non-integrated) expression vector or present in a viral vector genome that can be either integrating or non-integrating. The expression construct preferably comprises a suitable signal polypeptide which directs the target polypeptide to the outer cell membrane.

Examples of suitable signal polypeptides include those from: BM-40 (osteonectin SPARC), Vesicular Stomatitis Virus G (VSVG) protein, chymotrypsinogen, human interleukin-2 (IL-2), Gaussia luciferase, human serum albumin, influenza haemagglutinin and human insulin.

In some embodiments, the expression construct additionally comprises an inducible promoter element. Preferably, the inducible promoter element comprises a DNA sequence capable of binding proteins that can form a basal transcription complex and initiate transcription and a plurality of Tet operator sequences to which the Tet repressor protein (TetR) is capable of binding. In this bound state, tight suppression of transcription is obtained. However, in the presence of doxycycline, suppression is alleviated, thus allowing the promoter to gain full transcriptional activity. Such an inducible promoter element is preferably placed downstream of another promoter, e.g. the CMV promoter.

In some embodiments, the cells comprise multiple copies of the target polypeptide expression construct in order to increase the levels of target polypeptide which are expressed. Increasing levels of target polypeptide expression may also be achieved by increasing the time of cell culturing.

The target polypeptide expression construct may also comprise an antibiotic resistance gene, e.g. one encoding resistance to puromycin.

The target polypeptide is displayed on a population of mammalian cells. The cells may be isolated cells, e.g. they are not present in a living animal. Examples of mammalian cells include those from any organ or tissue from humans, mice, rats, hamsters, monkeys, rabbits, donkeys, horses, sheep, cows and apes. Preferably, the cells are human cells. The cells may be primary or immortalised cells. Preferred human cells include HEK293, HEK293T, HEK293A, PerC6, 911, HeLa and COS cells. Other preferred cells include CHO and VERO cells. Most preferably, the cells are CHO cells.

Preferably, all or substantially all of the cells in the population display the target polypeptide. Preferably, all or substantially all cells in the population express less than 10 or less than 5, more preferably 1, 2 or 3, and most preferably a single binding partner.

In the method of the invention, a library of binding partners is expressed in the population of mammalian cells. The aim is to identify at least one specific binding partner which binds to an exposed region or domain of the target polypeptide in such a way that cells to which such specific binding partners are bound can be identified.

As used herein, the term “specific binding partner” relates to the ability of the binding partner to bind to the target polypeptide with a desired degree of specificity and/or affinity. The specific binding partner might not bind exclusively to the target polypeptide. Preferably, a specific binding partner specifically binds if its affinity for its target polypeptide is about 5-fold greater than its affinity for a non-target polypeptide. Ideally, there is no significant cross-reaction or cross-binding with undesired substances.

The affinity of the specific binding partner may, for example, be at least about 5 fold, such as 10 fold, such as 25-fold, especially 50-fold, and particularly 100-fold or more, greater for a target molecule than its affinity for a non-target polypeptide.

In some embodiments, binding between a specific binding partner and a target polypeptide means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Antibodies may, for example, bind with an EC₅₀ of 50 nM or less, 10 nM or less, 1 nM or less, 100 pM or less, or more preferably 10 pM or less. The term “EC₅₀” as used herein, is intended to refer to the potency of a compound by quantifying the concentration that leads to 50% maximal response/effect. EC₅₀ may be determined by Scatchard or FACS.

Each binding partner comprises a core framework and a plurality of variable regions, each plurality of variable regions endowing that binding partner with a specific binding affinity for a target. The core framework may comprise one or more polypeptides. Preferably, there are 2-10, more preferably 2-6, 3-6, 4-6 or 5-6 variable regions. The binding partners will, in general, be polypeptides. These may or may not be glycosylated. The binding partners may be viewed as potential binding partners or potential specific binding partners of the target polypeptide.

The binding partners (e.g. antibodies or antibody mimetics) are secreted by or secreted from or secreted out of the cells from which they are produced. In some embodiments, the binding partners (e.g. antibodies or antibody mimetics) are secreted out of the cells from which they are produced and into the medium which surrounds the cells. In other terms, in this embodiment, the binding partners are released from the cells.

In other embodiments, the binding partners (e.g. antibodies or antibody mimetics) are secreted from the cells from which they are produced. The binding partners may or may not then be released into the medium which surrounds the cells.

Polypeptide binding partners (e.g. antibodies or antibody mimetics) and the target polypeptide will be synthesized in the mammalian cells by ribosomes which are attached to the rough endoplasmic reticulum (ER). These polypeptides will both comprise signal peptides in order to direct the passage of the polypeptides to the cell's secretory pathway. After they are synthesized, these polypeptides will translocate into the ER lumen, where they may be glycosylated and where molecular chaperones aid protein folding. Vesicles containing the polypeptides then enter the Golgi apparatus. In the Golgi apparatus, any glycosylation of the polypeptides may be modified and further posttranslational modifications, including cleavage and functionalization, may occur. The polypeptides are then moved into secretory vesicles which travel along the cytoskeleton to the edge of the mammalian cell. Further modification may occur in the secretory vesicles. Eventually, there is vesicle fusion with the cell membrane at a structure called the porosome, in a process called exocytosis, which results in release of the contents of the vesicle to the surrounding medium. Membrane-integrated proteins will be retained in the cell's plasma membrane when the vesicle contents are shed.

Since both polypeptide binding partners (e.g. antibodies or antibody mimetics) and target polypeptides are produced via this secretory pathway, it is possible that binding of some specific binding partners to the target polypeptide will occur during the course of this pathway. If this is the case, then the specific binding partner will not be secreted out of the cell into the external medium; it will remain bound to the target polypeptide. The specific binding partner and target polypeptide will therefore be presented—together—on the outer surface of the cell.

In some embodiments, the antibodies or antibody mimetics are secreted from the cells in which they are produced in a form wherein their CDR sequences are bound to the target polypeptide. In other embodiments, the antibodies or antibody mimetics are secreted from the cells in which they are produced in a form wherein their CDR sequences are not bound to the target polypeptide.

The binding partners (e.g. antibodies or antibody mimetics) are not covalently attached, directly or indirectly, to the surface of the cells. The binding partners (e.g. antibodies or antibody mimetics) are free to diffuse within the medium surrounding the cells (apart from those binding partners which bind to the target polypeptide within the secretory pathway).

As used herein, the term “library” refers to a plurality of (potential) binding partners, each having a different binding specificity and/or affinity. Each binding partner has a (common) core framework, and a plurality of different variable regions. Preferably, the term “library” refers to a plurality of polypeptides each having a different binding specificity and/or affinity. The plurality of polypeptides will, in general, be encoded by a plurality of polynucleotides.

Preferably, the library is delivered to the population of mammalian cell by a virus (preferably a retrovirus, more preferably a lentivirus) which is capable of infecting the mammalian cells.

In some preferred embodiments, polynucleotides coding for the binding partners polypeptides are expressed within the cells. These polynucleotides may be transiently expressed (e.g. from retroviral vectors) or expressed from expression vectors. In some embodiments, the expression vectors are integrated into the genomes of the cells.

In certain embodiments, the library of polypeptides may comprise at least 10⁶, 10⁷, 10⁸, 10⁹, 10¹⁰, 10¹¹, 10¹², 10¹³, 10¹⁴ or 10¹⁵ or more different polypeptides.

In some embodiments, the different polypeptides in the library are related through, for example, their origin from a single animal species (for example, human, mouse, rabbit, goat, horse), tissue type, organ or cell type.

In other embodiments, the library is a library of naturally-occurring polypeptides, which might be enriched. In yet other embodiments, the library is a library of synthetic polypeptides.

The binding partners must be capable of being secreted from the cells (preferably out of the cells). In some embodiments, such secretion may be aided by the inclusion of a 5′-signal polypeptide.

In some preferred embodiments, the binding partner is an antibody or an antibody mimetic. In this embodiment, Step (a) comprises expressing a library of antibodies or antibody mimetics in a population of mammalian cells, wherein each antibody or antibody mimetic is secreted from the cell in which it is produced.

“Antibody” as used herein includes a wide variety of structures, as will be appreciated by those in the art, that in some embodiments contain at a minimum a set of 6 CDRs, including, but not limited to traditional antibodies (including monoclonal antibodies), humanized and/or chimeric antibodies, antibody fragments, engineered antibodies, multi-specific antibodies (including bispecific antibodies), and other analogues known in the art.

An “antibody” is an immunoglobulin molecule which is capable of specific binding to a target, such as a carbohydrate, polynucleotide, lipid, polypeptide, etc., through at least one antigen recognition site, located in the variable region of the immunoglobulin molecule.

In some embodiments, the antibody can be a mixture from different species, e.g. a chimeric antibody and/or a humanized antibody. That is, the CDR sets can be used with framework and constant regions other than those from which they were originally obtained.

In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs.

In one embodiment, the antibody is an antibody fragment. Specific antibody fragments include, but are not limited to, (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al., 1989, Nature 341:544-546) which consists of a single variable region; (v) isolated CDR regions; (vi) F(ab′)₂ fragments, a bivalent fragment comprising two linked Fab fragments; (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883); (viii) bispecific single chain Fv (e.g. WO 03/11161); and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448).

The term “antibody” also includes domain antibodies, Nanobodies and UniBodies. The term “antibody” also includes fusion proteins comprising an antibody portion or fragment with an antigen recognition site. Most preferably, the antibody is a scFv antibody.

The antibody library may, for example, comprise immunoglobulin polypeptides of a certain type or class. For example, the library might encode antibody μ, γ, γ2, γ3, γ4, α1, α2, ε, or δ heavy chains, and/or antibody κ or λ light chains. The antibody isotypes may be IgM, IgD, IgG, IgA or IgE. Preferably, the antibodies are IgG1, IgG2, IgG3 or IgG4.

Although each member of any one library described herein may encode the same heavy or light chain constant region, the library may collectively comprise at least 10⁶, 10⁷, 10⁸, 10⁹, 10¹⁰, 10¹¹, 10¹², 10¹³, 10¹⁴ or 10¹⁵ or more different variable regions, i.e., a “plurality” of variable regions associated with a common constant region.

In one embodiment, the population of cells are produced by infecting (e.g. transiently infecting) an initial (homogenous) population of cells with a plurality of retroviral (preferably lentiviral) particles encoding a scFV library, wherein the scFv library comprises a plurality of scFV antibodies having different binding specificities and affinities.

For example, each retroviral particle may comprise a retroviral or lentiviral vector comprising a promoter, a signal peptide (to promote secretion of the scFv) and a scFv coding sequence. The vector may also comprise a 3′ tag, such as haemagglutinin, in order to aid recognition by secondary antibodies. The lentiviral vector (expression construct) may additionally comprise a polynucleotide sequence encoding an anti-apoptosis factor.

In a particularly preferred embodiment of the invention, an anti-apoptosis factor is inserted after (i.e. 3′) an IRES downstream (i.e. 3′) of the last stop codon of the nucleic acid encoding the target polypeptide. This provides a configuration where a promoter initiating transcription is upstream (i.e. 5′) to the coding sequence of the target polypeptide gene which is then followed (3′) by an IRES, which is then followed (3′) by the coding region for the anti-apoptosis factor gene. In this configuration, both the target polypeptide and anti-apoptosis factor are encoded by the same mRNA, but due to the relatively low efficiency of IRES-mediated translation, the target polypeptide will be translated in greater abundance than the anti-apoptosis factor.

The methods of the invention are not limited to methods involving antibodies. They may also be practised through the use of antibody mimetics. A wide variety of antibody mimetic technologies are known in the art. In particular, technologies such as Affibodies, DARPins, Anticalins, Avimers, and Versabodies that employ binding structures that, while they mimic traditional antibody binding, are generated from and function via distinct mechanisms.

Affibody molecules represent a new class of affinity proteins based on a 58-amino acid residue protein domain, derived from one of the IgG-binding domains of staphylococcal protein A. This three-helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55.). Further details of Affibodies and methods of production thereof may be obtained by reference to U.S. Pat. No. 5,831,012.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins are ubiquitous binding molecules, which occur, unlike antibodies, intra- and extra-cellularly. Their unique modular architecture features repeating structural units (repeats) which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains. Additional information regarding DARPins and other DRP technologies can be found in US 2004/0132028 and WO 02/20565.

Anticalins are an additional antibody mimetic technology. However, in this case, the binding specificity is derived from lipocalins, a family of low molecular weight proteins that are naturally and abundantly expressed in human tissues and body fluids.

Lipocalins have evolved to perform a range of functions in vivo associated with the physiological transport and storage of chemically sensitive or insoluble compounds. Lipocalins have a robust intrinsic structure comprising a highly conserved ß-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved ß-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains. Additional information regarding Anticalins can be found in U.S. Pat. No. 7,250,297 and WO 99/16873.

Another antibody mimetic technology useful in the context of the instant invention is Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets. Additional information regarding Avimers can be found in US 2006/0286603, 2006/0234299, 2006/0223114, 2006/0177831, 2006/0008844, 2005/0221384, 2005/0164301, 2005/0089932, 2005/0053973, 2005/0048512 and 2004/0175756.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

Given the structure of Versabodies, these antibody mimetics offer a versatile format that includes multi-valency, multi-specificity, a diversity of half-life mechanisms, tissue targeting modules and the absence of the antibody Fc region. Additional information regarding Versabodies can be found in US 2007/0191272.

In other embodiments, the binding partner is not an antibody or antibody mimetic For example, the desired specific binding partner of the target polypeptide may be a polypeptide ligand to which the target polypeptide is capable of binding. In such cases, the library of binding partners may be library of polypeptides whose amino acid sequences are based upon the amino acid sequence of a polypeptide ligand which is known to bind to the target polypeptide. For example, the polypeptides in such a library may have at least 60%, 70%, 80%, 90% or 95% amino acid sequence identity with the known polypeptide ligand.

Step (b) of the method of the invention comprises identifying and/or isolating cells within the population of mammalian cells to which specific binding partners are bound. (The specific binding partners will bind to the target polypeptides which are displayed on those cells.) The cells to which specific binding partners are bound are ones which produce specific binding partners of the target polypeptide. In this way, the desired specific binding partners of the target polypeptide can be identified and/or isolated.

The binding of the specific binding partner (e.g. antibody or antibody mimetic) to the target polypeptide may be detected by a number of different means, depending primarily on the identity of the specific binding partner. Such means are well known in the art and include the use of labelled secondary antibodies (e.g. fluorescent labels, biotin labels or radioactive labels), enzymatic means (e.g. colorimetric assays) and functional domains (e.g. polypeptide domains which promote or inhibit a certain activity).

In embodiments of the invention wherein the specific binding partner is an antibody or antibody mimetic, the binding of such antibodies or antibody mimetics to the target polypeptide may be detected by using a labelled secondary antibody. For example, if the specific binding partner is a full-length antibody, a labelled secondary antibody which binds to the Fc region of the primary antibody may be used. If the specific binding partner is an scFV antibody, a labelled secondary antibody which binds to a tag on the scFV antibody (e.g. a HA tag) may be used.

In some embodiments, the specific binding partner (e.g. primary antibody) or a secondary antibody which binds thereto comprises a functional domain whose presence and/or activity may be established and/or quantified. Examples of such functional domains include domains which promote or inhibit cell proliferation (e.g. appropriate domains of growth factors).

The method of the invention may be carried out in a liquid medium, in a semi-solid medium, in a solid medium (e.g. gel) or the cells may be fully or partially immobilised. Preferably, the method is carried out in a liquid medium, e.g. in an aqueous physiological medium, for example an aqueous physiological medium which is suitable for cell culture and for binding of potential specific binding partners to the target polypeptide. In this embodiment, the secreted binding partners will be in solution and hence they will be free to bind not only to the cells from which they were secreted, but also to other (e.g. neighbouring) cells. In such a case, the first cells within the population of cells to which specific binding partners are bound will be ones which produce that specific binding partner. Over time, the binding partners will be capable of contacting cells (e.g. by convecting or diffusing to them) other than those from which they were secreted, but in reasonably static systems the binding partners will bind first to cells from which they were secreted (if the binding partners are capable of binding to the target polypeptide).

If the method of the invention is carried out in liquid media, therefore, it may be necessary to assay the cells for any binding of specific binding partners at a number of different time points (e.g. 4 hours, 6 hours, 24 hours, 48 hours, etc.) in order to establish when the cells first become bound by internally-produced specific binding partners. Such cells may then be sorted or isolated (e.g. by fluorescent activated cell sorting (FACS) in the case of fluorescently-labelled specific binding partners).

In some embodiments, therefore, Step (b) comprises the step:
(b) identifying or isolating cells within the population of cells to which the specific binding partners first become bound.

In other embodiments, therefore, Step (b) comprises the step:
(b) identifying or isolating cells within the population of cells to which specific binding partners are bound after a time point when the specific binding partners may only be bound to target polypeptides which are displayed on cells from which the specific binding partners themselves have been secreted.

In light of the fact that, in most methods, there will be very few cells which secrete binding partners which are capable of binding to the target polypeptide, problems with the convection or diffusion of binding partners to other cells are not considered to be significant.

A further way to reduce the effects of such diffusion is to replace the liquid medium in a continuous or discontinuous manner, thus removing any diffusing binding partners from the liquid medium. In other embodiments, therefore, Step (a) additionally comprises the feature: wherein the method is carried out in a liquid medium which is replaced in a continuous or discontinuous manner.

In some embodiments of the invention, it is desirable to inhibit the movement of the binding partners (e.g. antibodies or antibody mimetics) away from the cells in which they are produced. This helps to reduce the level of non-specific background binding and to avoid the production of false-positive cells. One way to do this is to perform the method of the invention using a liquid medium whose dynamic viscosity is greater than that of water at 25° C. In this way, diffusion of binding partners (e.g. antibodies) away from the cells in which they are produced is reduced. The dynamic viscosity of water is 8.9×10⁻⁴ Pa·s at 25° C. Preferably, the dynamic viscosity of the liquid medium in which Step (a) of the method of the invention is carried out is at least 10×10⁻⁴ Pa·s at 25° C.

More preferably, the dynamic viscosity of the liquid medium in which Step (a) of the method of the invention is carried out is between 1×10⁻⁴ Pa·s and 10 Pa·s at 25° C., even more preferably between 0.01 Pa·s and 1 Pa·s at 25° C., and most preferably between 0.01 Pa·s and 0.1 Pa·s at25° C. For example, the dynamic viscosity of the liquid medium may be increased using a neutral viscosity increaser, e.g. a sugar, polyvinylpyrrolidine (PVP), polyethylene glycol (PEG, molecular weight up to 20 KDa, more preferably about 8 KDa, up to 50% vol/vol) or poly[N(2-hydroxypropyl)methacrylamide] (preferably 10-100 KDa, up to 40% wt/vol).

A further way to prevent diffusion of the binding partners away from the cells in which they are produced is to carry out the method of the invention in a gel. A gel is a solid jelly-like material that can have properties ranging from soft and weak to hard and tough. Gels are defined as a substantially dilute cross-linked system, which exhibits no flow when in the steady-state. By weight, gels are mostly liquid, yet they behave like solids due to a three-dimensional cross-linked network within the liquid. It is the crosslinking within the fluid that gives a gel its structure (hardness) and contributes to the adhesive stick (tack). In this way, gels are a dispersion of molecules of a liquid within a solid in which the solid is the continuous phase and the liquid is the discontinuous phase. Preferably, the gel is a hydrogel, i.e. a cross-linked network of hydrophilic polymer chains.

In some embodiments, the hydrogel is formed from polyvinyl alcohol, sodium polyacrylate, acrylate polymers, or polymers or copolymers with an abundance of hydrophilic groups such as copolymers based on poly[N(2-hydroxypropyl)methacrylamide] or block copolymers based on polyethylene glycol or oligopeptides. In other embodiments, the hydrogel is formed from alginate, agarose, methylcellulose, hyaluronan, or other naturally-derived polymers.

Preferably, the gel is an alginate hydrogel, more preferably a calcium alginate hydrogel. Cells which are entrapped within such a gel may readily be released upon application of a divalent cation chelator, e.g. EDTA or EGTA, and then the ‘labelled’ cells may be isolated (e.g. by flow sorting) in the normal way. The hydrogel may be in the form of beads.

The background level of non-specific binding of the binding partners (e.g. antibodies or antibody mimetics) to the cells can be reduced by a negative selection step. In this step, cells to which binding partners are (non-specifically) bound before the target polypeptide is displayed on the cells are first removed from the cell population. In some embodiments, therefore, Step (a) comprises:
(a1) expressing a library of binding partners in a population of cells, wherein each binding partner is secreted from the cell in which it is produced,
(a2) removing cells from the population of cells to which binding partners bind; and then
(a3) inducing expression (preferably from an inducible promoter) of the target polypeptide on the outer surface of each cell in the population of cells.

Preferably, the inducible promoter is one which comprises a plurality of Tet operator sequences to which the Tet repressor protein (TetR) is capable of binding. In the bound state, tight suppression of transcription is obtained. Step (a3) may comprise the additional step of contacting the cells with doxycycline (which displaces the Tet repressor proteins, thus allowing the promoter to gain full transcriptional activity).

Once the cells which produce specific binding partners (e.g. antibodies or antibody mimetics) of the target polypeptides have been identified (i.e. those to which the specific binding partner bind), those cells may be purified, by any suitable means. Preferably, the cells to which the specific binding partners (e.g. antibodies) bind are purified by flow cytometry (e.g. FACS).

In some embodiments, the cells to which the specific binding partners (e.g. antibodies) bind are purified more than once (i.e. reiteratively) by flow cytometry (e.g. FACS).

The polynucleotides which encode the specific binding partners (e.g. antibodies or antibody mimetics) which are produced by the purified cells may be sequenced, thus providing the amino acid sequence of all or part of the specific binding partners. Preferably, the amino acid sequences of one or more of the antibody's or antibody mimetic's CDR sequences are obtained.

The amino acid sequences of the most promising specific binding partners may then be subjected to affinity maturation, e.g. by mutagenesis of the amino acid sequence, in order to produce specific binding partners (e.g. antibodies or antibody mimetics) with higher affinity or specificity for the target polypeptide.

In a further embodiment, the invention provides a specific binding partner (e.g. an antibody or antibody mimetic) which has been identified by a method of the invention.

In yet a further embodiment, the invention provides a process for producing a population of cells, the process comprising the steps: transforming a first population of mammalian cells with
(a) a plurality of first expression constructs, the plurality of first expression constructs encoding a library of secretable binding partners (e.g. antibodies or antibody mimetics); and
(b) a second expression construct which encodes a desired target polypeptide

the target polypeptide comprising a transmembrane domain, so as to produce a second population of mammalian cells, wherein each cell in the second population of mammalian cells secretes or is capable of secreting one or more binding partners (e.g. antibodies or antibody mimetics), and wherein each cell in the second population of mammalian cells displays or is capable of displaying the target polypeptide on the outer surface of the mammalian cell.

As used herein, the term “transforming” refers to any step by which the expression constructs are inserted into the cells. Hence it includes any form of electroporation, conjugation, infection (e.g. via retroviral particles) or transfection, inter alia.

Preferably, the binding partners are antibodies, more preferably scFV antibodies, or antibody mimetics (preferably DARPins). The library of first expression constructs preferably encodes an antibody library as defined herein.

Preferably, the term “transforming a population of cells with a plurality of first expression constructs” comprises infecting the population of cells with a plurality of retroviral particles (preferably lentiviral particles) encoding a scFV library, wherein the scFv library comprises a plurality of scFV antibodies having different binding specificities and/or affinities.

Each cell (or substantially each cell) in the second population of cells secretes or is capable of secreting one or more binding partners (e.g. antibodies or antibody mimetics). Preferably, each cell (or substantially each cell) in the population of cells secretes or is capable of secreting 1-3, 1-2 or most preferably just 1 binding partner (e.g. antibody).

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims. The disclosure of each reference set forth herein is incorporated herein by reference in its entirety.

### Example 1: Demonstration of Self-Labelling by Cells Expressing Anti-EpCAM Antibodies

Epithelial cell adhesion molecule (EpCAM) was selected as a suitable target polypeptide (bait antigen). EpCAM is a glycosylated, 30- to 40-kDa type I membrane protein containing three potential N-linked glycosylation sites.

HEK293 cells were transfected with an EpCAM-expression construct (HEK293 cells are normally EpCAM negative) together with a secreted HA-tagged anti-EpCAM single chain antibody expression-construct. After a 24-hour incubation period, the cells were stained with a fluorescently-labelled anti HA-tag antibody and analysed by flow cytometry to determine which cells had self-labelled their membrane EpCAM with the encoded anti-EpCAM antibody.

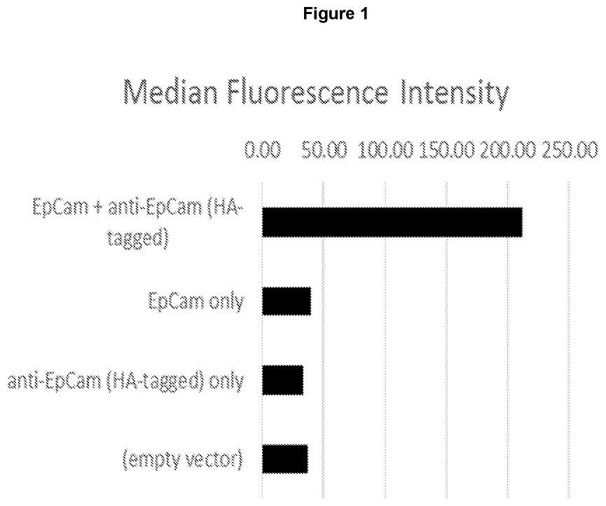

The results are shown in FIG. 1. Cells expressing both the ‘bait’ antigen (EpCAM) and the scFv became highly fluorescent, whereas all of the other cells (expressing either EpCAM only or the scFv only) did not. This shows that cells secreting scFv can label antigens on their own surface.

### Example 2: Optimising Stringency to Prevent Labelling of Irrelevant Cells

Two populations of EpCAM-expressing cells were made. Some expressed the anti-EpCAM antibodies, while others instead expressed green fluorescence protein (GFP). Cells were mixed at different ratios (always with the antibody-expressing cells in the minority), incubated for different times, fixed and surface-bound antibody was visualised by staining in the red channel. The small number of cells expressing the antibody invariably labelled themselves first, giving rise to cells in the lower right hand quadrant of the flow cytometry plot (cells were red but not green, indicating that the antibody-producing cells were labelled before the irrelevant cells).

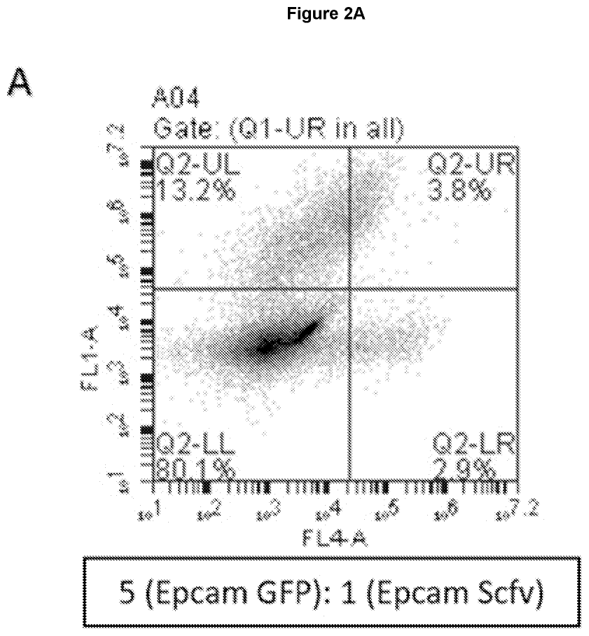

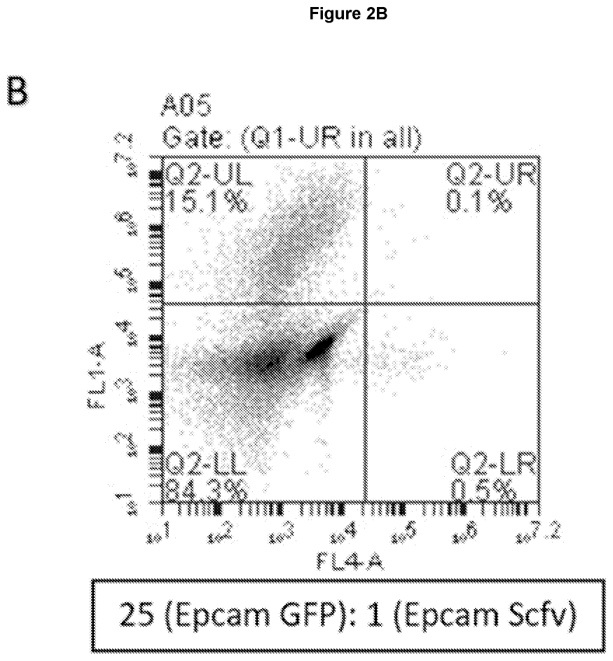

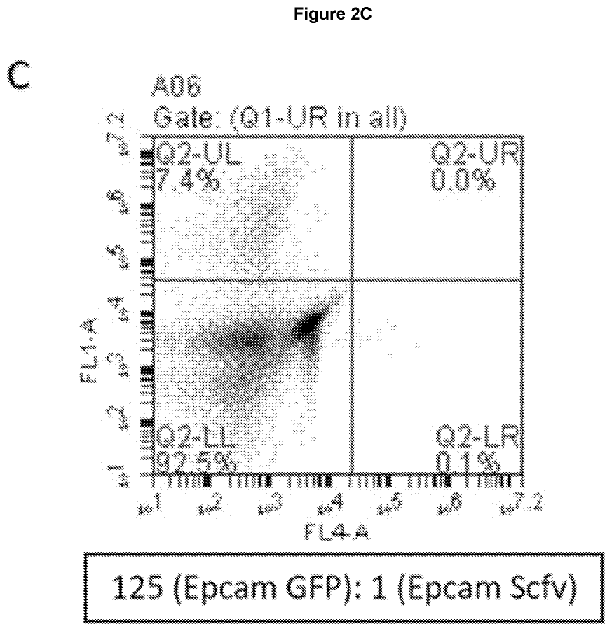

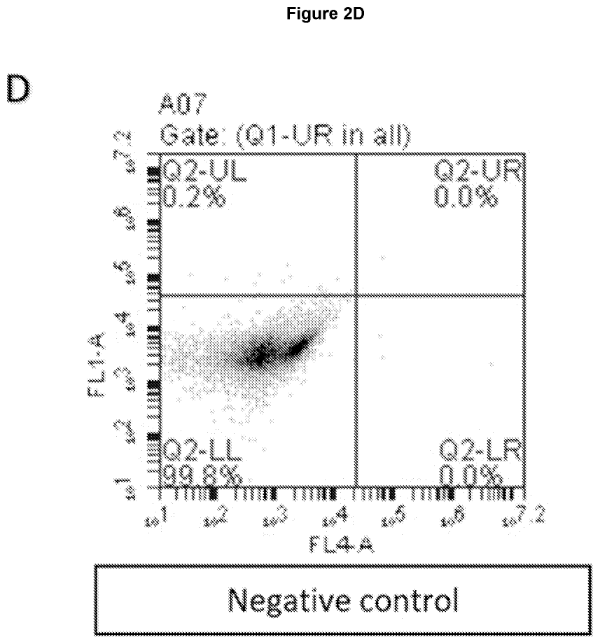

The results are shown in FIG. 2. Even at a dilution of 1:125, an appreciable number of cells appear in the lower right quadrant, representing cells expressing antibodies that bind the cell-surface antigen.

### Example 3: Production of Antibodies to DRD1

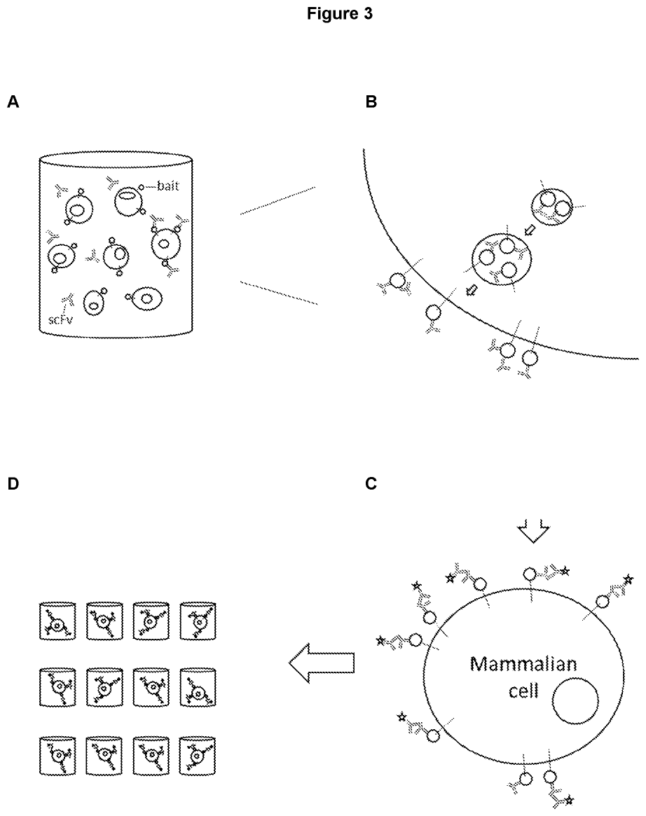

In this example, the target polypeptide (bait) is DRD1; this is expressed in CHO cells (see FIG. 3 for an overview). A retrovirus transfer vector is used to clone the target polypeptide (bait) construct and the antibody libraries into the CHO cells.

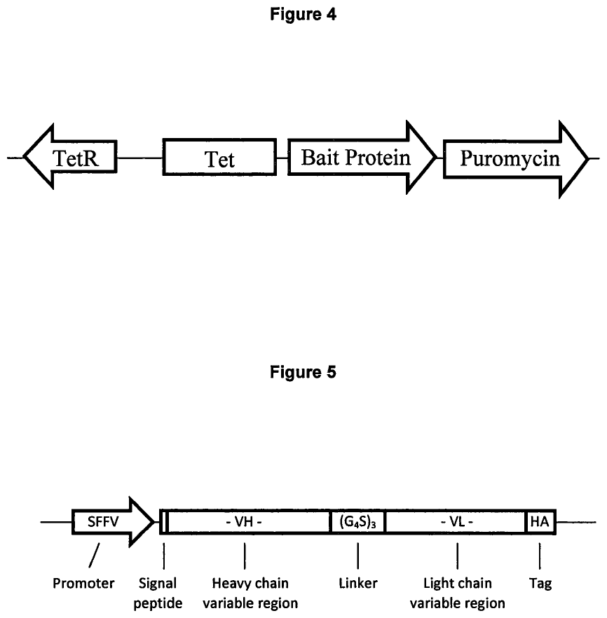

The target polypeptide (bait) cell lines are produced by using the retrovirus system to integrate the gene for the target polypeptide (bait) into the host cell (CHO) genome along with a selectable marker (see FIG. 4). The target polypeptide construct also contains the gene for the Tet repressor (TetR). Target polypeptide (bait) expression is driven by a doxycycline-inducible promoter.

A library of retrovirus particles encoding a cDNA-based library of human scFv sequences of human-like scFv sequences is used to infect the CHO cells. For the scFv libraries, the retrovirus transfer vector is modified to contain a constitutive promoter (SFFV) and the flanking regions of the scFv antibody subunits (see FIG. 5). Each scFV also includes a HA tag.

After a 24-hour incubation period, the cells are stained with a fluorescently-labelled anti HA-tag antibody and analysed by flow cytometry to determine which cells had self-labelled their membrane DRD1 with a scFV antibody.

1. A method of identifying a cell which produces an antibody or antibody mimetic which binds to a target polypeptide, the method comprising the steps:
(a) expressing a library of antibodies or antibody mimetics in a population of mammalian cells, wherein each antibody or antibody mimetic is secreted from the cell in which it is produced, wherein the target polypeptide is displayed on the an outer surface of each cell in the population of mammalian cells and wherein the target polypeptide is an integrated membrane protein; and
(b) isolating cells within the population of mammalian cells to which the antibodies or antibody mimetics are bound,
wherein the cells to which the antibodies or antibody mimetics are bound are ones which produce the antibodies or antibody mimetics which bind to the target polypeptide.

2. The method as claimed in claim 1, wherein the target polypeptide is expressed within each cell in the population of cells, preferably from an expression construct.

3. The method as claimed in claim 1, which additionally comprises the step:
(c) sequencing (preferably all or part of) the polynucleotide sequences in the isolated cells which encode the antibodies or antibody mimetics which bind to the target polypeptide.

4. The method as claimed in claim 1, wherein the target polypeptide is an immune checkpoint molecule.

5. The method as claimed in claim 1, wherein the mammalian cells are selected from the group consisting of those from any organ or tissue from humans, mice, rats, hamsters, monkeys, rabbits, donkeys, horses, sheep, cows and apes, preferably human cells.

6. The method as claimed in claim 1, wherein the antibody is an scFV antibody.

7. The method as claimed in claim 1, wherein the antibody mimetic is an Affibody, DARPin, Anticalin, Avimer or Versabody, preferably a DARPin.

8. The method as claimed in claim 1, wherein the antibody or antibody mimetic is detected using a labelled secondary antibody which binds to the antibody or antibody mimetic.

9. The method as claimed in claim 1, wherein the method is carried out in a liquid medium, in a semi-solid medium, in a solid medium or the cells are fully or partially immobilised.

10. The method as claimed in claim 1, wherein Step (b) comprises the step:
(b) isolating cells within the population of cells to which the antibodies or antibody mimetics first become bound.

11. The method as claimed in claim 1, wherein Step (b) comprises the step:
(b) isolating cells within the population of mammalian cells to which antibodies or antibody mimetics are bound after a time point when the antibodies or antibody mimetics may only be bound to target polypeptides which are displayed on cells from which the antibodies or antibody mimetics have been secreted.

12. The method as claimed in claim 1, wherein Step (a) additionally comprises the feature:
wherein the method is carried out in a liquid medium which is replaced in a continuous or discontinuous manner.

13. The method as claimed in claim 1, wherein the dynamic viscosity of the liquid medium in which Step (a) is carried out is at least 10×10⁻⁴ Pa·s at 25° C.

14. The method as claimed in claim 1, wherein the medium in which Step (a) is carried out is a hydrogel, preferably an alginate gel.

15. The method as claimed in claim 1, wherein Step (a) comprises:
(a1) expressing a library of antibodies or antibody mimetics in a population of mammalian cells, wherein each antibody or antibody mimetic is secreted from the cell in which it is produced,
(a2) removing cells from the population of mammalian cells to which antibodies or antibody mimetics bind; and then
(a3) inducing expression (preferably from an inducible promoter) of the target polypeptide on the outer surface of each cell in the population of mammalian cells.

16. The method as claimed in claim 15, wherein the inducible promoter is one which comprises a plurality of Tet operator sequences to which the Tet repressor protein (TetR) is capable of binding.

17. The method as claimed in claim 1, additionally comprising the step of sequencing (preferably all or part of) nucleic acid in the cell which encodes the antibody or antibody mimetic in order to obtain a nucleotide sequence of (preferably all or part of) the antibody or antibody mimetic which binds to the target polypeptide.

18. The method as claimed in claim 1, additionally comprising the steps of purifying the antibody or antibody mimetic, and sequencing (preferably all or part of) the purified antibody or antibody mimetic to obtain the amino acid sequence of (preferably all or part of) the antibody or antibody mimetic which binds to the target polypeptide.

19. (canceled)

20. A process for producing a population of mammalian cells, the process comprising the step:
transforming a first population of mammalian cells with:
(a) a plurality of first expression constructs, the plurality of first expression constructs encoding a library of secretable antibodies or antibody mimetics; and
(b) a second expression construct which encodes a desired target polypeptide, the target polypeptide comprising a transmembrane domain, so as to produce a second population of mammalian cells, wherein each cell in the second population of mammalian cells secretes or is capable of secreting one or more antibodies or antibody mimetics, and wherein each cell in the second population of mammalian cells displays or is capable of displaying the target polypeptide on an outer surface of the mammalian cell.

21. The process as claimed in claim 20, wherein the target polypeptide is an integrated membrane protein.

22. (canceled)

23. (canceled)
